(19) United States
(12) Patent Application Publication
(10) Pub. No.: US 2006/0024386 A1
(43) Pub. Date: Feb. 2, 2006

(54) Herbal extract with antiviral properties

(76) Inventor: Jacob J. Abdullahi, Garki (NG)

Correspondence Address: KENYON & KENYON, 1500 K STREET NW, SUITE 700, WASHINGTON, DC, 20005, US

(21) Appl. No.: 11/189,802

(22) Filed: Jul. 27, 2005

Related U.S. Application Data
(60) Provisional application No. 60/591,939, filed on Jul. 29, 2004.

(51) Int. Cl. A61K 36/185

(52) U.S. Cl. 424/725; 424/769

(57) ABSTRACT

Herbal extract compositions designed and used for the clinical management of HIV/AIDS, especially in sub-Saharan Africa and Nigeria. Herbal extract compositions are any one of or any combination of Ficus exaspirata, Ficus asperifolia, Ficus sur and Sida acuta/Sida corymbosa. Methods of treating HIV/AIDS infection with herbal extract compositions are also provided.

### CROSS-REFERENCE TO RELATED APPLICATIONS

The present application claims priority to U.S. Provisional Application No. 60/591,939, filed on Jul. 29, 2004, which is incorporated by reference herein.

### FIELD OF THE INVENTION

The present invention is directed to a composition comprising a herbal antiviral extract. The composition may be used to treat human immunodeficiency virus (HIV) and acquired immune deficiency syndrome (AIDS) infection.

### BACKGROUND

In 2004, nearly 40 million people globally were estimated to be living with HIV. An estimated 25 million people were living with HIV in sub-Saharan Africa and rates of infection are still on the rise. In fact, since the first clinical evidence of HIV in Nigeria in 1986, HIV seroprevalence has steadily risen from 0.1% in 1989 to 5.8% in 2002. Despite these rising numbers in certain parts of the world, treatment of patients infected with HIV has changed enormously in some countries. For example, new AIDS cases and deaths began to decline in incidence in the United States since 1996. The advent of more potent agents, particularly availability of inhibitors of HIV-1 protease, has been a major contributor to this decline. Use of potent combination therapy (also known as highly active antiretroviral therapy or “HAART”) has been associated with dramatic decreases in incidence of new opportunistic events.

A major dilemma confronting patients and practitioners, however, is that antiretroviral regimens currently available that have the greatest potency in terms of viral suppression and CD4+ T-cell preservation are medically complex. They are also associated with a number of side effects and drug interactions and pose a substantial challenge for adherence. In sub-Saharan Africa, these problems are further compounded by poverty. Unequal access to affordable treatment and adequate health services is one of the main factors accounting for drastically different survival rates among those living with HIV/AIDS in rich and poor countries and communities.

As such, there is a need for an HIV/AIDS treatment that is accessible, affordable, and has minimal side effects.

### SUMMARY OF THE INVENTION

In an embodiment, the present invention provides a composition comprising Ficus exaspirata extract, Ficus asperifolia extract, Ficus sur extract, Sida corymbosa extract, Sida acuta extract, or any combination thereof. In a preferred embodiment, the present invention provides a composition comprising Ficus exaspirata and any one or any combination of Ficus asperifolia extract, Ficus sur extract, Sida corymbosa extract, or Sida acuta extract. In a more preferred embodiment, the present invention provides a composition comprising Ficus exaspirata extract, Ficus asperifolia extract, Ficus sur extract, and at least one of Sida corymbosa extract or Sida acuta extract.

In another embodiment, the present invention provides a method of treating HIV infection by administering to a patient with HIV a compositio'n comprising Ficus exdspirata extract, Ficus asperifolia extract, Ficus sur extract, Sida corymbosa extract, Sida acuta extract, or any combination thereof. In a preferred embodiment, the method of treating HIV infection comprises administering Ficus exaspirata extract and any one or any combination of Ficus asperifolia extract, Ficus sur extract, Sida corymbosa extract, or Sida acuta extract. In a more preferred embodiment, the method of treating HIV infection comprises administering a composition comprising Ficus exaspirata extract, Ficus asperifolia extract, Ficus sur extract, and at least one of Sida corymbosa extract or Sida acuta extract.

### DETAILED DESCRIPTION OF INVENTION

Ficus exaspirata, Ficus asperifolia, Ficus sur, Sida acuta, and Sida corymbosa are all sub-Saharan tropical herbs found in the middle belt of the Federal Republic of Nigeria and along the West African Coastal Region. The physiochemical properties of herbal extract compositions of the present invention comprising any one of or any combination of the aforementioned herbs reveal a dark brown crystalline powder, with a strong pungent leafy smell, bland taste and a fine-granular texture. The compositions are freely soluble in water with a moisture content of 0.1%. Phytochemical screening of the compositions indicate the presence of alkaloids, tannins, saponins, and complex polysaccharides and the absence of cyanogenic glycosides.

Herbal extract compositions of the present invention are used to treat HIV infection (including AIDS) in an HIV-infected patient. In a preferred embodiment, the herbal extract compositions of the present invention treat HIV infection by increasing CD⁺4 T lymphocyte count, reducing HIV viral load, alleviating one or more symptoms of HIV infection, increasing hemoglobin count, increasing packed cell volume, increasing total white blood cell count, or any combination thereof of the patient. Alleviating one or more of the symptoms of HIV infection includes alleviating fever, weight loss, diarrhea, persistent cough, oral thrush, rash, itching or any combination thereof of the patient.

Herbal extracts of the present invention can be administered to the patient as part of a pharmaceutical composition that contains herbal extracts of the present invention as the active agent or a pharmaceutically acceptable salt, hydrate, or pro-drug thereof, in combination with a pharmaceutically acceptable carrier. “Pharmaceutically acceptable carrier” includes any and all solvents excipients, preservatives, stabilizers, dispersion media, coatings, wetting agents (for example, sodium lauryl sulfate), isotonic and absorption delaying agents, disintrigrants (for example, potato starch or sodium starch glycolate), and the like. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®., polyethylene glycol (PEG), and PLURONICS®.

Herbal extract compositions of the present invention including any pharmaceutical compositions comprising the herbal extracts of the present invention can be administered to a patient by any method that may result in treating the HIV infection. For example, the herbal extract compositions may be delivered to a patient orally, parenterally (intramuscularly or intravenously), transdermally, or by inhalation. The term patient as used herein means a mammal and the term mammal is intended to include, but is not limited to, humans, laboratory animals, domestic pets and farm animals.

For oral administration, a herbal extract composition may be administered, for example, in liquid form with an inert diluent or assimilable carrier, or incorporated into a solid dosage form. Examples of oral liquid and solid dosage forms include, for example, solutions, suspensions, syrups, emulsions, tablets, lozenges, capsules (including soft gelatin capsules) and the like. Oral dosage forms may be formulated as sustained release products using, for example, a coating to delay disintegration or to control diffusion of the herbal extract composition.

For intravenous or intramuscular administration, a herbal extract composition may be administered in an injectable dosage such as sterile liquids or solids. Sterile liquids include solutions, emulsions and suspension and sterile solids include sterile powders that are reconstituted, dissolved or suspended in a liquid prior to injection. Carriers of such injectable dosages include sterile water, saline, injectable organic esters, peanut oil, vegetable oil, and the like. Such sterile formulations can be prepared by heating, irradiation, microfiltration, and/or by addition of Various antibacterial and antifungal agents such as, for example, parabens, chlorobutanol, phenol, sorbic acid, thimerosal, and the like.

For transdermal administration a herbal extract composition of the present invention may be incorporated in a gel or matrix base for application in a patch, optionally providing controlled release of the pharmaceutical composition containing the herbal extract composition through a transdermal barrier. Herbal extract compositions of the present invention can also be formulated by known methods for rectal administration.

Suitable dosages of the herbal extract compositions of the present invention can be determined by a physician or qualified medical professional depending on factors such as the severity of the HIV/AIDS infection, the route of administration, the duration of treatment, and the condition of the patient. The herbal extract compositions of the present invention may be administered as frequently as necessary in order to obtain the desired therapeutic effect of HIV/AIDS treatment. Frequency of administration will depend, for example, on the nature of the dosage form used and the stage of infection. In a preferred embodiment, 480 milligrams of a herbal composition of the present invention comprising Ficus exaspirata extract, Ficus asperifolia extract, Ficus sur extract, and at least one of Sida corymbosa extract or Sida acuta extract is administered twice a day.

In another embodiment, the present invention provides a method of treating HIV infection by administering to a patient with HIV a composition comprising Ficus exaspirata extract, Ficus asperifolia extract, Ficus sur extract, Sida corymbosa extract, Sida acuta extract, or any combination thereof. In a preferred embodiment, the method of treating HIV infection comprises administering Ficus exaspirata extract and any one or any combination of Ficus asperifolia extract, Ficus sur extract, Sida corymbosa extract, or Sida acuta extract. In a more preferred embodiment, the method of treating HIV infection comprises administering a composition comprising Ficus exaspirata extract, Ficus asperifolia extract, Ficus sur extract, and at least one of Sida corymbosa extract or Sida acuta extract. In a preferred embodiment, a method of treating HIV infection according to the present invention includes increasing CD⁺4 T lymphocyte count, reducing HIV viral load, alleviating one or more symptoms of HIV infection, increasing hemoglobin count, increasing packed cell volume increasing total white blood cell count, or any combination thereof of the patient. Alleviating one or more of the symptoms of HIV infection include alleviating fever, weight loss, diarrhea, persistent cough, oral thrush, rash, itching or any combination thereof of the patient.

With respect to a method of making a herbal extract composition of the present invention, in an embodiment, Ficus exaspirata, Ficus asperifolia, Ficus sur, Sida corymbosa, and Sida acuta extracts are harvested in dry season, sun-dried, grinded and combined in a ratio of 1:5 to distilled water. Each of the herbal extracts of the compositions of the present invention can be a liquid extract such an aqueous extract. The liquid extract can also be a dry liquid extract. Preferably, the Ficus exaspirata extract Ficus asperifolia extract, Ficus sur extract, and at least one of Sida corymbosa extract or Sida acuta extract are present in a herbal composition of the present invention in a ratio of about 0.5-5:0.1-1.0:0.1-1.0:0.1:1.0. In amore preferred embodiment, the Ficus exaspirata extract, Ficus asperifolia extract, Ficus sur extract, and at least one of Sida corymbosa extract or Sida acuta extract are present in the herbal composition in a ratio of about 5:1:1:1.

### EXAMPLES

### Example 1

The present example shows treatment of HIV infection by administering a composition comprising Ficus exaspirata extract, Ficus asperifolia extract, Ficus sur extract, and at least one of Sida corymbosa extract or Sida acuta extract to treat HIV infection.

Clinical and laboratory evaluations were comprehensively conducted on twenty three (n=23) individuals who had received a full course of treatment with a composition of the present invention. Specifically, each individual received one teaspoon (5 ml) of a liquid suspension of a composition comprising Ficus exaspirata extract, Ficus asperifolia extract, Ficus sur extract, and at least one of Sida corymbosa extract or Sida acuta extract in a ratio of 5:1:1:1 (156.25 mg/5 ml, 31.25 mg/5 ml, 31.25 mg/5 ml, 31.25/5 ml, respectively) (referred to in this example as “the composition”) taken two times daily for a period of eight weeks.

TABLE 1
Demographics

| # of | | | | | |
|---|---|---|---|---|---|
| Individuals | Age | # of Males | # of Females | # Married | # Single |
| 23 | 21-50 | 14 | 9 | 16 | 7 |

The history of other drugs ingested along with the composition of the present example in the course of treatment was also obtained. Samples of urine and blood were collected from each individual for analysis. Blood samples were assessed for screening and confirmatory test for HIV-1 and -2 (all 23 individuals were confirmed to have HIV-1), hemoglobin levels, packed cell volume, white blood cells count (total and differential), urea, electrolytes, liver function tests, and CD4+ count. Body weights and vital signs were also recorded.

Table 2 shows the clinical presentation before and after treatment with the composition of the present example. The presenting symptoms before treatment were fever, weight loss, diarrhea, persistent cough, tuberculosis, oral thrush, herpes, rashes and itching. After treatment, there was a marked reduction in prevalence of symptoms. Out of the 15 people (65.22%) that presented with fever and diarrhea before treatment, symptoms persisted in only 5 people (21.74%) after treatment. The prevalence of weight loss among the individuals was also reduced from 9 (39.13%) before treatment to 1 (4.4%) after treatment.

TABLE 2
Clinical Presentation Before and After Treatment

| | | Weight | | Persistant | | Oral | | | |
|---|---|---|---|---|---|---|---|---|---|
| | Fever | Loss | Diarrhea | Cough | Tuberculosis | Thrush | Herpes | Rash | Itching |
| Before | 65.2 | 39.1 | 65.2 | 39.1 | 8.7 | 13.0 | 4.4 | 17.4 | 8.7 |
| (%) | | | | | | | | | |
| After | 21.7 | 4.4 | 21.7 | 43.8 | — | 8.7 | 4.4 | 30.4 | 4.3 |
| (%) | | | | | | | | | |

Baseline CD4 assessment was performed for four out of the 23 individuals before commencement of treatment. Table 3 shows the mean CD4 count before and after treatment of these four individuals. As seen from Table 3, after treatment with the composition of the present example, there was a 202.8% increase in CD4 count.

TABLE 3
CD4 Count (cells/ul) Before and After Treatment

| Mean CD4 Count Before | Mean CD4 Count After | % Increase in |
|---|---|---|
| Treatment | Treatment | CD4 Count |
| 170.5 | 516.3 | 202.8 |

Table 4 shows hematological measurements before and after treatment. Specifically, Table 4 shows hemoglobin count, packed cell volume count, white blood cell count (total), neutrophil count, lymphocyte count, monocyte count, and eosinophil count. As seen from Table 4, there was an improvement in the value of some of the hematological parameters after treatment. For example, the mean hemoglobin count increased by 22.43%, the mean packed cell volume increased by 30.92%, and the white blood cell count increased by 30.92%.

TABLE 4
Measurement of Hematological Parameters Before
and After Treatment

| | | | Standard | | |
|---|---|---|---|---|---|
| | | | Error | Standard | |
| | Mean | Mean | of Mean | Error of | |
| | Count | Count | Count | Mean Count | |
| | Before | After | Before | After | |
| Parameter | Treatment | Treatment | Treatment | Treatment | P-Value |
| Hemoglobin | 10.03 | 12.28 | 0.45 | 0.52 | 0.01 |
| Packed Cell | 30.25 | 37.63 | 1.36 | 1.81 | <0.01 |
| Volume | | | | | |
| White Blood | 3.04 | 3.98 | 0.34 | 0.39 | <0.01 |
| Cell | | | | | |
| Neutrophil | 37.06 | 44.41 | 2.78 | 2.75 | <0.1 |
| Lymphocyte | 53.94 | 48.35 | 2.65 | 0.33 | 0.1 |
| Monocyte | 5.56 | 1.63 | 0.65 | 0.33 | <0.01 |
| Eosnophil | 3.56 | 5.35 | 0.47 | 1.04 | >0.1 |

### Example 2

The present example shows treatment of HIV infection by administering a composition comprising Ficus exaspirata extract, Ficus asperifolia extract, Ficus sur extract, and at least one of Sida corymbosa extract or Sida acuta extract to treat HIV infection.

Clinical and laboratory evaluations were comprehensively conducted on twelve (n=12) individuals who had received a full course of treatment with a composition of the present invention. Specifically, each individual received one teaspoon (5 ml) of a liquid suspension of a composition comprising Ficus exaspirata extract, Ficus asperifolia extract, Ficus sur extract, and at least one of Sida corymbosa extract or Sida acuta extract in a ratio of 5:1:1:1 (156.25 mg/ml, 31.25 mg/5 ml, 31.25 mg/5 ml, 31.25/5 ml, respectively) (referred to in this example as “the composition”) taken two times daily for a period of eight weeks.

Table 5 shows the demographic data of individuals for the study.

TABLE 5
Demographics

| # of Individuals | Age | # Married | # Single |
|---|---|---|---|
| 12 | 26-52 | 8 | 4 |

Table 6 shows the clinical presentation before and after treatment with the composition of the present example. The presenting symptoms before treatment were fever, weight loss, diarrhea, persistent cough, tuberculosis, herpes, rashes and itching. After treatment, there was a marked reduction in prevalence of symptoms such as reduction in fever, weight loss, diarrhea, and persistant cough.

TABLE 6
Clinical Presentation Before and After Treatment

| | | Weight | | Resistant | | | | |
|---|---|---|---|---|---|---|---|---|
| | Fever | Loss | Diarrhea | Cough | Tuberculosis | Herpes | Rash | Itching |
| Before | 58.3. | 58.3 | 50 | 33.3 | 16.7 | 16.7 | 8.3 | 16.7 |
| (%) | | | | | | | | |
| After | 33.3 | 16.7 | 16.7 | 16.7 | 16.7 | 16.7 | 8.3 | 16.7 |
| (%) | | | | | | | | |

Baseline CD4 assessment was performed on all twelve individuals. Table 7 shows the mean CD4 count before and after treatment of these individuals. As seen from Table 7, after treatment with the composition of the present example, there was a 41.6% increase in CD4 count.

TABLE 7
CD4 Count (cells/ul) Before and After Treatment

| Mean CD4 Count Before | Mean CD4 Count After | % Increase in |
|---|---|---|
| Treatment | Treatment | CD4 Count |
| 709 | 1214 | 41.6 |

Table 8 shows results of liver and renal function tests. As seen from Table 8, treatment with the composition did not significantly affect liver and renal function (P=0.05).

TABLE 8
Liver and Renal Function

| | Meas- | Standard | Meas- | Standard | |
|---|---|---|---|---|---|
| | urement | Deviation | urement | Deviation | |
| | Before | Before | After | After | Normal |
| Parameter | Treatment | Treatment | Treatment | Treatment | Range |
| HCO₃ | 24 | 2.4 | 23 | 2.6 | 24-31 |
| (mmol/L) | | | | | |
| Cl⁻ | 100 | 1.7 | 97 | 1.8 | 98-107 |
| (mmol/L) | | | | | |
| Na⁺ | 140 | 3.4 | 135 | 3.0 | 135-145 |
| (mmol/L) | | | | | |
| K⁺ | 5.0 | 0.0 | 6.3 | 2.58 | 3.5-5.1 |
| (mmol/L) | | | | | |
| SGOT (U/L) | 40 | 15.3 | 34 | 44.0 | 8-20 |
| SGPT (U/L) | 31.5 | 15.15 | 28.6 | 40.94 | 10-40 |
| ALP (U/lL) | 61 | 18.8 | 27.2 | 15.22 | 38-94 |
| Cr (mmol/L) | 1.1 | 0.34 | 1.1 | 0.16 | 62-115 |
| Protein (g/L) | 99.4 | 18.0 | 64.7 | 38.02 | 68-83 |
| Albumin | 34.1 | 6.7 | 38 | 7.3 | 35-52 |
| (g/L) | | | | | |
| Urea | 16.2 | 6.7 | 13.5 | 6.65 | 2.1-7.1 |
| (mmol/L) | | | | | |
| Bilirubin | 0.1 | 0.04 | 0.2 | 0.11 | Up to 4.3 |
| (mmol/L) | | | | | conjugated |
| Bilirubin | 0.4 | 0.34 | 0.3 | 0.12 | Up to 18.8 |
| (mmol/L) | | | | | total |

### Example 3

The present example shows treatment of HIV infection by administering a composition comprising Ficus exaspirata extract, Ficus asperifolia extract, Ficus sur extract, and at least one of Sida corymbosa extract or Sida acuta extract to treat HIV infection.

A clinical trial was undertaken, over an 8-week period, using a Randomized Double Blind Placebo with two groups of asymptomatic HIV-confirmed individuals: a study group of 53 individuals designated as Group Y and a control group of 50 individuals designated as Group X. The clinical trial was conducted at the Winners Medical Diagnostic/Research Institute, Plot 1391 Ladongor Close, Garki II, Abuja, Nigeria. Each individual in each group received one teaspoon of a liquid suspension of a composition comprising Ficus exaspirata extract, Ficus asperifolia extract, Ficus sur extract, and at least one of Sida corymbosa extract or Sida acuta extract in a ratio of 5:1:1:1 (156.25 mg/5 ml, 31.25 mg/5 ml, 31.25 mg/5 ml, 31.25/5 ml, respectively) (referred to in this example as “the composition”) taken two times daily for a period of eight weeks. Vital signs, viral load and CD4⁺-T lymphocytes, count, hematological parameters, and liver and renal function was assessed for each individual in each group.

The vital sign assessment of Groups Y and X before and after treatment is summarized in Tables 9 and 10, respectively. As seen from Table 9, in Group Y, the mean axial temperature before treatment with the composition was 35.78° C. and 36.27° C. after treatment. After treatment, the systolic blood pressure of Group Y was reduced from 117.71 to 106.02 mmHg and the diastolic from 72 to 65.25 mmHg. The pulse rate and respiration rate remained stable after treatment, while the weight slightly varied from 63.59 to 62.59 Kg.

As seen from Table 10, in Group X, the mean axial temperature was slightly reduced after placebo administration from 35.98° C. to 36.44° C., the systolic pressure equally reduced from 115.16 to 112.71 mmHg after placebo administration while the diastolic pressure increased from 72.52 to 73.94 mmHg. There was no significant difference on pulse rate and respiratory rate. The weight also remained stable.

TABLE 9
Vital Sign Assessment of Group Y

| | Before Therapy | | After Therapy | |
|---|---|---|---|---|
| N = 53 | | Standard | | Standard |
| Parameters | Mean | Deviation | Mean | Deviation |
| Axial | 35.78 | 0.85 | 36.27 | 0.62 |
| Temperature | | | | |
| (° C.) | | | | |
| Blood Pressure | 117.71/72.21 | 20.64/16.64 | 106.02/65.25 | 14.75/10.32 |
| (mmHg) | | | | |
| Pulse Rate | 76.46 | 18.43 | 76.73 | 16.48 |
| (/min) | | | | |
| Respiratory | 22.31 | 2.42 | 22.04 | 1.57 |
| Rate (/min) | | | | |
| Weight (Kg) | 63.59 | 13.68 | 62.59 | 13.74 |

TABLE 10
Vital Sign Assessment of Group X

| | Before Therapy | | After Therapy | |
|---|---|---|---|---|
| N = 50 | | Standard | | Standard |
| Parameters | Mean | Deviation | Mean | Deviation |
| Axial | 35.98 | 0.79 | 36.44 | 0.54 |
| Temperature | | | | |
| (° C.) | | | | |
| Blood Pressure | 115.16/72.52 | 18.63/14.39 | 112.71/73.94 | 18.61/17.76 |
| (mmHg) | | | | |
| Pulse Rate | 79.66 | 18.96 | 78.26 | 13.66 |
| (/min) | | | | |
| Respiratory | 22.60 | 1.91 | 22.29 | 1.22 |
| Rate (/min) | | | | |
| Weight (Kg) | 63.68 | 12.80 | 63.75 | 12.74 |

Tables 11 and 12 show the results of viral RNA load assessment before and after treatment for Group Y and Group X, respectively. As seen from Table 11, the mean viral RNA copies of Group Y were 29,233 copies/mL before treatment with the composition of the present example and 16,065 copies/mL after treatment. As seen from Table 12, the mean viral RNA copies of Group X, on the other hand, was 17,634 copies/mL before placebo administration and 28,597 copies/mL after placebo administration.

TABLE 11
Viral Load Assessment of Group Y

| | Before Therapy | After Therapy |
|---|---|---|
| Total Viral Load (cp/ml) | 1,549,350 | 851,430 |
| Mean Viral Load (cp/ml) | 29,233 | 16.065 |
| Standard Error of Mean | 9,696 | 4,873 |
| Standard Deviation | 70,585 | 35,474 |
| Calculated t Value | 2.3 | 2.3 |
| Tabulated t Value | 2.0 | 2.0 |

TABLE 12
Viral Load Assessment of Group X

| | Before Therapy | After Therapy |
|---|---|---|
| Total Viral Load (cp/ml) | 881,690 | 1,429,860 |
| Mean Viral Load (cp/ml) | 17,634 | 28,597 |
| Standard Error of Mean | 6,677 | 6,902 |
| Standard Deviation | 47,215 | 48,807 |
| Calculated t Value | 4.1 | 4.1 |
| Tabulated t Value | 2.0 | 2.0 |

A summary of the virologic response in the study Group Y and control Group X is shown in Tables 13 and 14, respectively. As indicated in these tables, in the study group Y, 46 individuals (86.8%) had a viral load reduction following treatment with the composition and 10 of these (18.9%) had undetectable viral RNA copies after 8 weeks of treatment. Out of those with reduced viral levels after treatment, 30 (56.5%) had viral reduction of more than 50% relative to their baseline values, while six (11%) recorded viral reduction below 50% relative to baseline. Among the study individuals in Group Y, seven (13.2%) did not show viral reduction after 8 weeks of treatment (virologic failure). In the control-Group X, on the other hand, 48 of the 50 individuals (96%) showed a viral RNA increase after placebo administration. Two individuals (4%) showed viral reduction, one of which was more than 50% reduction in viral load relative to the baseline values.

It is noted that a minimally significant change in viremia is considered a 3 fold or 0.5 log₁₀ increase, and this is achieved in 4 weeks following initiation of conventional antiretroviral therapy, or less than a 1 log₁₀ reduction in eight weeks. See Lucas G M, Chaesson R E, More R D, “Highly active antiretroviral therapy in a large urban clinic: Risk factors for virologic failure and adverse drug reactions,” Ann intern Mid, 1999, 131: P 81-87, which is incorporated by reference herein. Therefore, the composition could be said to have achieved a virologic success of about 57% (individuals with more than 50% viral RNA reduction relative to their baseline). Further, whereas non-detectable viral levels are usually achieved after about 6-12 months of therapy, the present example shows that undetectable viral RNA levels were achieved in about 19% of individuals receiving treatment with the composition after only eight weeks of treatment.

It should also be noted that although as many as 70-90% of anti-retroviral drug-naïve individuals achieve maximal viral load suppression 6-12 months after initiation of therapy, only about 50% of individuals in a city clinic setting achieved similar results. See Ledergerber B, Egger M, Opravd M et al, “Clinical progression and virological failure on highly active antiretroviral therapy in HIV-1 patients: A prospective cohort study Swiss HIV Cohist study.” Lancet, 1999.3533: P 863-868; William G. P. Alan L. Michael M. L. “Recovery of the Immune System with antiretroviral therapy: the End of opportunism?” JAMA 1998, 280:72-77. both of which are incorporated by reference herein. Predictors of virologic success include low baseline viremia and high baseline CD4⁺ T cell count. See Fischl M A, Richman O D. Grieco M H. et al. “The efficacy of azidothymidine (AZT) in the treatment of patients with AIDS and AIDS-related complex,” N Engl J Med 1998; 317:185-191, which is incorporated by reference herein. In this present example, non-detectable viral load was achieved in individuals in Group Y with baseline viral loads of 6000 copies/ml or less.

TABLE 13
Summary of Virologic Response in Study Group Y

| Virologic Response (number of individuals) | Percentage |
|---|---|
| Viral RNA load reduction after therapy (46) | 86.8% |
| Undetectable viral RNA load (10) | 18.9% |
| More than 50% viral RNA load reduction (30) | 56.6% |
| Less than 50% viral RNA load reduction (6) | 11% |
| Virologic failure (7) | 13.2% |

TABLE 14
Summary of Virologic Response in Control Group X

| Virologic Response (number of individuals) | Percentage |
|---|---|
| Viral RNA load increase after therapy (48) | 96% |
| Viral RNA load reduction (2) | 4% |
| More than 50% viral RNA load reduction (1) | 2% |
| Less than 50% viral RNA load reduction (1) | 2% |

As seen from Tables 15 and 16, the mean CD4⁺-T lymphocyte count of Group Y was found to be 413 cells/μl before treatment with the composition and increased to 521 cells/μl after 8 weeks of treatment, while the mean CD4⁺ count of Group X before placebo administration was 431 cells/μl and 418 cells/μl after placebo administration. Therefore, statistical analysis showed a significant statistical difference in the CD4⁺-T lymphocytes counts of Group Y as compared to Group X, as the mean CD4⁺ count of Group Y increased by more than 100 cells/μl after 8 weeks of treatment.

TABLE 15
Analysis of Variance of Mean CD4+ T-Cell Counts
Before and After Treatment for Group Y

| | | | | | Error of |
|---|---|---|---|---|---|
| | Number of | Mean CD4+ | Standard | Standard | t-Value |
| | Individuals | Count | Deviation | Mean | df = 49 |
| Before | 50 | 413 | 233.69 | 33.05 | 0.3456 |
| Treatment | | | | | |
| After | 50 | 521 | 305.61 | 43.22 | 1.5158 |
| Treatment | | | | | |

TABLE 15
Analysis of Variance of Mean CD4+ T-Cell Counts
Before and After Treatment for Group X

| | | | | | Error of |
|---|---|---|---|---|---|
| | Number of | Mean CD4+ | Standard | Standard | t-Value |
| | Individuals | Count | Deviation | Mean | df = 49 |
| Before | 50 | 431 | 284.58 | 40.25 | 0.3456 |
| Treatment | | | | | |
| After | 50 | 418 | 370.78 | 52.44 | 1.5158 |
| Treatment | | | | | |

Therefore, the composition of the present example has some immunologic effects in HIV individuals as demonstrated by the average increase in CD4⁺T⁻ lymphocytes count of about 100 cells/μl in the individuals in study Group Y as opposed to individuals receiving placebo administration in control Group X.

Table 17 shows the comparative analyses of various hematological parameters between Group Y and Group X. As seen from Table 17, no significant variation was found in all the parameters except the total white blood cell count and the percentage granulocyte count.

TABLE 17
Comparative Analysis of Hematological Parameters Between Group Y and
Group X

| | | BEFORE THERAPY | | | | | AFTER THERAPY | | | | |
|---|---|---|---|---|---|---|---|---|---|---|---|
| | | X GROUP | | Y GROUP | | | X GROUP | | Y GROUP | | |
| PARAMETERS | UNITS | X | SD | X | SD | t-TEST | X | SD | X | SD | t-TEST |
| Total white blood | ×10⁹/L | 4.10 | 3.37 | 4.10 | 3.74 | N.S | 3.5 | 3.34 | 5.26 | 3.40 | S |
| cell | | | | | | | | | | | |
| Lymphocytes | % | 46.2 | 10.89 | 50.7 | 13.72 | N.S | 44.5 | 13.08 | 47.9 | 12.18 | N.S |
| Granulocytes | % | 49.3 | 9.78 | 45.3 | 12.99 | N.S | 49.5 | 13.00 | 44.9 | 12.04 | S |
| Monocytes | % | 4.9 | 2.60 | 4.5 | 2.24 | N.S | 5.6 | 3.24 | 6.3 | 7.03 | N.S |
| Granulocytes | ×10⁹/L | 2.8 | 0.77 | 3.1 | 3.12 | N.S | 2.8 | 1.27 | 2.7 | 0.90 | N.S |
| Lymphocytes | ×10⁹/L | 2.6 | 0.87 | 3.3 | 1.17 | S | 2.8 | 1.34 | 3.90 | 2.49 | N.S |
| Monocytes | ×10⁹/L | 0.1 | 0.18 | 0.2 | 0.19 | S | 0.2 | 0.22 | 0.2 | 0.7 | N.S |
| Red Blood Cells | ×10⁹/L | 4.7 | 0.56 | 4.5 | 0.53 | S | 4.7 | 0.67 | 4.5 | 0.67 | N.S |
| Haemoglobin | G/dl | 13.1 | 1.66 | 12.7 | 1.24 | N.S | 13.0 | 2.13 | 12.5 | 1.74 | N.S |
| Hamatocrit | % | 37.0 | 4.44 | 37.7 | 3.51 | N.S | 38.8 | 5.54 | 37.5 | 4.97 | N.S |
| Mean Cell Volume | Fl | 81.4 | 13.46 | 84.0 | 5.73 | N.S | 83.7 | 7.09 | 84.0 | 5.79 | N.S |
| Mean Cell | Pg | 27.9 | 2.75 | 28.2 | 2.24 | N.S | 27.8 | 2.75 | 28.0 | 2.28 | N.S |
| Haemaglobin | | | | | | | | | | | |
| Mean Cell | | | | | | | | | | | |
| Haemaglobin | | | | | | | | | | | |
| Concentration | G/dl | 33.5 | 0.97 | 33.5 | 0.81 | N.S | 33.2 | 0.78 | 33.3 | 0.83 | N.S |
| Red Cell | % | 14.0 | 1.31 | 14.3 | 1.51 | N.S | 15.3 | 1.69 | 14.9 | 1.97 | N.S |
| Distribution width | | | | | | | | | | | |
| Platelets | ×10⁹/L | 177.6 | 85.31 | 167.1 | 87.87 | N.S | 201.3 | 87.11 | 189.3 | 108.5 | N.S |
| Mean Platelets | Fl | 8.1 | 0.94 | 7.8 | 0.97 | N.S | 7.1 | 1.02 | 7.2 | 0.84 | N.S |
| Volume | | | | | | | | | | | |
| Plateletcrit | % | 0.15 | 0.07 | 0.14 | 0.07 | N.S | 0.15 | 0.06 | 0.13 | 0.07 | N.S |
| Platelet | | 16.9 | 1.19 | 16.7 | 1.08 | N.S | 16.7 | 1.21 | 16.6 | 1.21 | N.S |
| Distribution Width | | | | | | | | | | | |

S = Significant
N.S = Non-significant

Therefore, the hematological parameters assessed in the present example were observed to be generally within the reference ranges used. This is most probably because individuals were asymptomatic and might not have had their hemopoietic system impaired significantly by HIV-infection or its sequalae. This is also in consonant with earlier findings that there are no specific abnormal haematologic features in early/latent period of HIV-infections. See Kaslov R. A, Phair J. P., Friedman H B, et al (1987) “Infection with the human immunodeficiency virus clinical manifestation and their relationship to immune deficiency. A report from the Multicenter AIDS Cohort study.” Ann Intern Med; 80:286-93, which is incorporated by reference herein.

Table 18 shows the assessment of liver function in Group Y and Group X. The laboratory parameters used for liver function assessment in this study were all within reference ranges before and after treatment and no statistically significant difference was found in the outcome of the Group Y when compared Group X as seen in Table 18.

The renal function assessment test results for Group Y were also found to be within normal reference ranges and did not vary significantly when compared to the outcome of that of the control Group X, before and after treatment, as seen in Table 19.

TABLE 18
Liver Function Assessment Parameters for Group Y

| | | BEFORE | | AFTER | | |
|---|---|---|---|---|---|---|
| | | THERAPY | | THERAPY | | |
| Parameters (n = 50) | (Units) | MEAN (x) | SD* | MEAN (x) | SD* | t-TEST |
| Alanine Transferase | U/l | 29.41 | 7.89 | 31.25 | 7.41 | Not significant |
| Aspartate Transferase | U/l | 33.24 | 11.38 | 37.20 | 13.27 | Not significant |
| Alkaline Phosphatase | U/l | 104.06 | 44.45 | 115.00 | 46.98 | Not significant |
| γ-glutamyl transferase | U/l | 25.15 | 15.94 | 29.92 | 14.50 | Not significant |
| Total Bilirubin | mg/dl | 13.26 | 4.99 | 12.53 | 5.15 | Not significant |
| Conjugated Bilirubin | mg/dl | 5.33 | 2.08 | 5.26 | 2.00 | Not significant |
| Total Protein | g/L | 68.31 | 10.61 | 69.65 | 6.75 | Not significant |
| Albumin (ALB) | g/L | 41.92 | 3.69 | 41.80 | 3.42 | Not significant |
| Globulin (GLO) | g/L | 27.32 | 6.54 | 27.87 | 6.37 | Not significant |
| ALB+/GLO+ RATIO | — | 1.66 | 0.59 | 1.60 | 0.49 | Not significant |

*SD = Standard Deviation

TABLE 17
Renal Function Assessment Parameters for Group Y

| | | BEFORE | | AFTER | | |
|---|---|---|---|---|---|---|
| | | THERAPY | | THERAPY | | |
| Parameters (n = 50) | Units | MEANS (x) | SD* | MEANS (x) | SD* | t-TEST |
| Sodium (Na+) | mmol/l | 138.10 | 4.63 | 137.81 | 4.03 | Not significant |
| Potassium (K+) | mmol/l | 4.09 | 0.68 | 4.19 | 0.66 | Not significant |
| Chloride (Cl−) | mmol/l | 101.04 | 4.52 | 102.98 | 4.2 | Not significant |
| Bicarbonate (HCO-3) | mmol/l | 23.17 | 1.78 | 22.90 | 1.1 | Not significant |
| Urea | mmol/l | 4.15 | 1.36 | 4.28 | 1.21 | Not significant |
| Creatinine | mmol/l | 61.71 | 18.50 | 63.27 | 19.35 | Not significant |
| Uric acid | mmol/l | 200.14 | 66.28 | 215.18 | 69.30 | Not significant |

Therefore, the composition of the present example did not result in any untoward effects on the liver and renal functions, at least for the period studied. The tests results did not vary significantly before and after drug therapy and the results were also within reference ranges.

### Example 4

The present example describes sustained HIV viral inhibition after treatment with a composition comprising Ficus exaspirata extract, Ficus asperifolia extract, Ficus sur extract, and at least one of Sida corymbosa extract or Sida acuta extract (referred to as “the composition” in the present example).

The long term effect of the composition of the present example on serum viral load was assessed by re-evaluating the serum RNA copies of HIV/AIDS Individuals (n=21), who had undergone the herbal treatment until non-detectable viral loads was achieved and had been off the herbal treatment for at least, six months, using Nucleic Acid Sequence Based Amplification (NASBA) assay. Results obtained indicate that 80% (n=16) of the individuals had non-detectable viral RNA copies while 10% (n=2), had detectable viral RNA copies, three (3) of the individuals had invalid results. These differences were statistically significant at 0.05 confidence limit, (p-value <0.001).

The foregoing description and examples have been set forth merely to illustrate the invention and are not intended as being limiting. Each of the disclosed aspects and embodiments of the present invention may be considered individually or in combination with other aspects, embodiments, and variations of the invention. In addition, unless otherwise specified, none of the steps of the methods of the present invention are confined to any particular order of performance. Modifications of the disclosed embodiments incorporating the spirit and substance of the invention may occur to persons skilled in the art and such modifications are within the scope of the present invention. Furthermore, all references cited herein are incorporated by reference in their entirety.

I claim:

1. A composition comprising Ficus exaspirata extract in an amount effective to treat HIV infection.

2. The composition of claim 1, wherein the Ficus exaspirata extract is administered in combination with Ficus asperifolia extract, Ficus sur extract, and at least one of Sida corymbosa extract or Sida acuta extract, wherein each of said extracts is present in said composition in an amount effective in combination with the other extracts in treating HIV infection.

3. The composition of claim 1 comprising Sida corymbosa extract.

4. The composition of claim 1 comprising Sida acuta extract.

5. The composition of claim 1 comprising both Sida corymbosa extract and Sida acuta extract.

6. The composition of claim 1, wherein each of said extracts comprises a liquid extract.

7. The composition of claim 1, wherein treating HIV infection comprises increasing CD+4 T lymphoctye count in a individual.

8. The composition of claim 1, wherein treating HIV infection comprises reducing HIV viral load in the individual.

9. The composition of claim 1, wherein treating HIV infection comprises alleviating one or more symptom of HIV infection in the individual.

10. The composition of claim 9, wherein said symptom of HIV comprises one or more of fever, weight loss, diarrhea, persistent cough, oral thrush, rash, or itching in the individual.

11. The composition of claim 1, wherein treating HIV infection comprising increasing the hemoglobin count of the individual.

12. The composition of claim 1, wherein treating HIV infection comprises increasing the packed cell volume of the individual.

13. The composition of claim 1, wherein treating HIV infection comprises increasing the total white blood cell count of the individual.

14. The composition of claim 1 further comprising a pharmaceutically acceptable carrier or diluent.

15. A unit dosage form comprising the composition of any of claim 1.

16. The unit dosage form of claim 14 comprising a tablet, capsule or caplet.

17. The composition of claim 2, wherein the Ficus exaspirata extract, Ficus asperifolia extract, Ficus sur extract, and at least one of Sida corymbosa extract or Sida acuta extract are present in the ratio of about 0.5-5:0.1-1:0.1-1:0.1-1.

18. The composition of claim 16, wherein the wherein the Ficus exaspirata extract, Ficus asperifolia extract, Ficus sur extract, and at least one of Sida corymbosa extract or Sida acuta extract are present in the ratio of about 5:1:1:1.

19. A method for treating HIV infection comprising administering to a individual with HIV the composition of claim 1.

20. The method of claim 18, wherein treating HIV infection comprises increasing CD+4 T lymphoctye count in a individual.

21. The method of claim 18, wherein treating HIV infection comprises reducing HIV viral load in the individual.

22. The method of claim 18, wherein treating HIV infection comprises alleviating one or more symptom of HIV infection in the individual.

23. The method of claim 21, wherein said symptom of HIV comprises one or more of fever, weight loss, diarrhea, persistent cough, oral thrush, rash, or itching in the individual.

24. The method of claim 18, wherein treating HIV infection comprising increasing the hemoglobin count of the individual.

25. The method of claim 19, wherein treating HIV infection comprises increasing the packed cell volume of the individual.

26. The method of claim 19, wherein treating HIV infection comprises increasing the total white blood cell count of the individual.
